(19) United States
(12) Patent Application Publication
(10) Pub. No.: US 2013/0105577 A1
(43) Pub. Date: May 2, 2013

(54) TRACABILITY TAG SORTER

(71) Applicants: Barry Hildreth, Southaven, MS (US); Madonna Paul, Memphis, TN (US); Lora Fusco, Cordova, TN (US)

(72) Inventors: Barry Hildreth, Southaven, MS (US); Madonna Paul, Memphis, TN (US); Lora Fusco, Cordova, TN (US)

(73) Assignee: WARSAW ORTHOPEDIC, INC., Warsaw, IN (US)

(21) Appl. No.: 13/284,599

(22) Filed: Oct. 28, 2011

(51) Int. Cl. G06Q 90/00; B65D 85/00

(52) U.S. Cl. 235/385; 206/307

(57) ABSTRACT

A system and method for tracking inventory levels and uses of a medical device. The system includes one or more medical devices having tags associated therewith, in which the tags include indicia containing information regarding the medical device to which it is attached, and at least one tag includes a tamper evident locking mechanism. The system also includes a storage device having a plurality of pockets of sufficient size and shape to receive at least one tag after it has been separated from the medical device to which it was previously associated. The plurality of pockets may he arranged to correspond to the position in the anatomy where the medical device is intended to be implanted, and each pocket is equipped with indicia to indicate the position in the anatomy where the medical device is intended to be implanted.

### BACKGROUND

There is a need to track medical devices from their base materials and manufacture to their use, as well as after use in a surgical setting. These include sterile and non-sterile medical devices. Anon-sterile medical device is a medical device that is shipped from a manufacturer in a condition that is not adequately sterilized for implantation. A sterile medical device is shipped from the manufacture in a condition adequately sterilized for implantation. Healthcare providers may prefer to receive non-sterile medical devices for various reasons. In that regard, because non-sterile devices can be sterilized onsite before a medical procedure, non-sterile medical devices having a longer shelf life than a corresponding sterile medical device. Furthermore, non-sterile medical devices typically are less expensive to package. Additionally, non-sterile medical devices typically can be more densely packaged into a common carrier than sterile devices.

Difficulty arises in tracking these medical devices. For example, some smaller medical devices are difficult to track because medical devices generally do not have adequate surface area for applying marks. Thus, in many instances, medical devices are not tracked beyond their manufacturing facility, and may only be counted when reconciled for payment as one of many products that were not returned to a manufacturer for replenishment.

It is known to place a tag or tags on medical devices that are intended to be implanted into the body. For example, U.S. Patent Application Publication No. 2008/0230423, the disclosure of which is incorporated herein by reference in its entirety, discloses a holding device for an implant, such as a screw or rod to be used in surgery, where the holding device may contain identifying information regarding the implant. Other tags attached to medical devices, particularly small medical devices are disclosed in co-pending U.S. patent application Ser. No. 12/109,517, filed Apr. 25, 2008, U.S. patent application Ser. No. 12/109,534, tiled Apr. 25, 2008, U.S. patent application Ser. No. 12/109,539, filed Apr. 25, 2008, U.S. patent application Ser. No. 12/512,274, filed Jul. 30, 2009, and U.S. patent application Ser. No. ______, entitled: “Encapsulated Data Carrier Tag for Track and Trace Purposes,” docket number P0041164, the disclosures of each of which are entirely incorporated herein by reference.

While these tags provide a means for tracking and tracing medical devices, once the devices are removed from the tags, it is not always easy to determine which device was implanted in which position in the body during the surgery. The tags could be scanned after surgery and information regarding where the implant was implanted in the patient can be read into the tag. This becomes very difficult during complicated surgical procedures in which many implants are used in the surgery, such as spinal surgeries in which large deformities are treated. Accordingly, devices, systems and methods for tracking and tracing medical devices are needed.

### SUMMARY

Disclosed herein is a tag storage system and method for tracking tags associated with medical devices in which the tags are separated from the medical device when the device is selected for use during a surgery. The system described in one embodiment is comprised of a storage device for storing a plurality of tags after removal of one or more medical devices from the tag. The storage device includes a plurality of pockets of sufficient size and shape to receive the removed tag in which the pockets are arranged to correspond to the position in the anatomy where the device is intended to be implanted. Each pocket is equipped with indicia to indicate the position in the anatomy where the device is intended to be implanted.

In accordance with another feature of an embodiment, there is provided a method of tracking a medical device that includes providing a plurality of medical devices associated with an identification tag. The method also includes providing a storage device for storing a plurality of tags after removal of one or more medical devices from the tag. The method further includes removing at least one medical device from its associated tag, and placing the tag previously associated with that at least one medical device in the storage device in the position in the storage device corresponding to the position in the anatomy where the at least one medical device was implanted. The method concludes by correlating the information concerning each medical device with its position in the anatomy, and optionally updating the patient record to indicate where each medical device was implanted.

The system and method described herein enables tracking of medical devices from manufacture all the way through use. The system and method also provides a fast and easy way to document the position where each medical device is placed on a patient, and store the information regarding the medical device, its position, and the patient into which the device(s) has been implanted in a retrievable and searchable database. In accordance with the system and method described herein, a medical device manufacturer can track a medical device throughout its entire life, thereby providing useful information for product development, product recalls, and the like. These and other aspects, forms, objects, features, and benefits of the present invention will become apparent from the following detailed drawings and description.

### BRIEF DESCRIPTION OF THE DRAWINGS

In the accompanying drawings, which are incorporated in and constitute a part of the specification, embodiments of the invention are illustrated, which, together with a general description of the invention given above, and the detailed description given below, serve to exemplify the embodiments of this invention.

FIG. 1 is a perspective view of a tag for attachment to a medical device according to one embodiment of the present disclosure.

FIG. 2 is a perspective view of a tag that has been removed from a medical device in accordance with one embodiment.

FIG. 3 is a plan view of a storage device for storing a plurality of tags after they have been removed from their associated medical device in accordance with an embodiment of the invention.

FIG. 4 is an enlarged view of FIG. 3 as outlined in A in FIG. 3.

### DETAILED DESCRIPTION

The present disclosure relates generally to the field of orthopedic surgery, and more particularly to devices, systems and methods for tracking and tracing medical devices through the use of removable tags. For the purposes of promoting an understanding of the principles of the invention, reference will now be made to embodiments or examples illustrated in the drawings, and specific language will be used to describe these examples. It will nevertheless be understood that no limitation of the scope of the invention is thereby intended. Any alteration and further modifications in the described embodiments, and any further applications of the principles of the invention as described herein, are contemplated as would normally occur to one skilled in the art to which the disclosure relates.

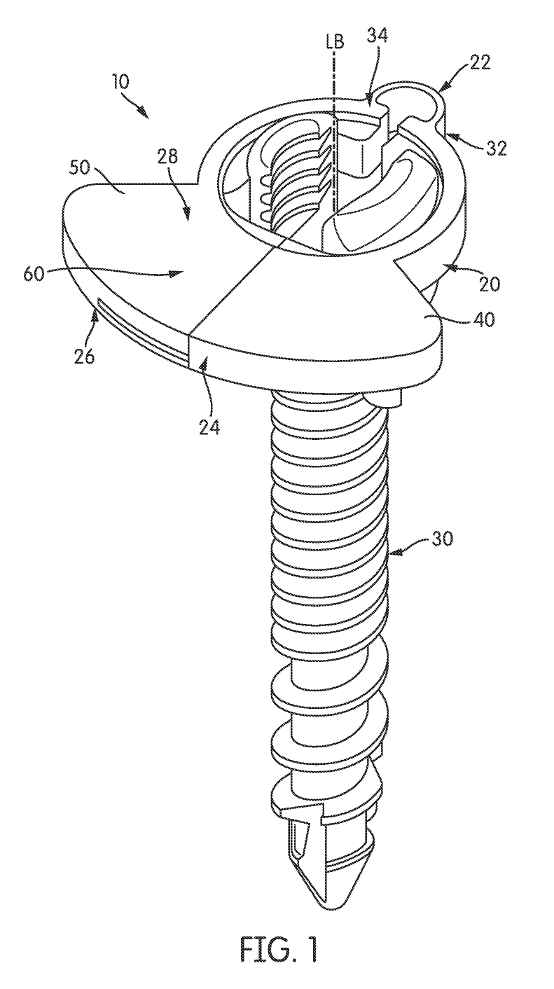

Referring first to FIG. 1, a perspective view of a medical device 30 associated with a data carrier, or tag 20 is shown, in which the combined product is referenced by numeral 10. The tag is shown in its locked position with lobes 40 and 50 connected to one another. The medical device 30 can be inserted into the tag 20 when lobes 40 and 50 are spread apart, and then the two lobes 40, 50 can be brought into engagement with one another. The lobes 40, 50 remain connected to one another until the medical device 30 is removed, the connection being any connection means, including adhesive, snap fit, mechanical fastening with a screw, nail, rivet, or the like. The tag 20 typically is provided with a tamper evident locking mechanism that allows the medical device 30 to be removed upon application of force, such as twisting the tag 20, or snapping lobes 40, 50 in opposing directions along longitudinal axis LB.

The tag 20 includes a distal portion 22 and proximal portion 24, although their relationship as distal and proximal can be reversed. Distal portion includes opposing surfaces 32, 34, and the proximal portion includes opposing surfaces 26, 28. Tag 20 also includes an enlarged substantially planar portion 60, which as shown in FIG. 1 is essentially a combination of lobes 40 and 50. Enlarged substantially planar portion 60 may include indicia that represent markings or tracking devices capable of retaining identifying information, for example, relevant to the medical device 30 captured by tag 20. Additionally, the indicia may represent identifying information related to the patient receiving the device, medical procedure used with the device, manufacturing information such as materials, processes, customer/supplier, and lot information of similarly manufactured devices. The identifying information is not intended to be limiting in scope by this disclosure, but instead is presented for exemplarily purposes only. Furthermore, any required manufacturing standards requiring the marking of products with certain identifying information is considered to be within the scope of identifying information capable of be represented by the indicia. Therefore, there is no need for the medical device 30 captured by tag 20 to have identifying information because the tag 20 provides any necessary identifying information.

In the embodiment shown in FIG. 1, the indicia represent a two dimensional bar code and alphanumeric lettering, respectively used to identify the medical device 30 captured by tag 20. However, the indicia, or any tracking device herein, may be any device that is capable of retaining identifying information. For example, the indicia can be a one or two dimensional barcode capable of being scanned by an optical scanner. Such an optical scanner may include a barcode scanner made by Baracoda such as the Evolution scanner (part number: B40160202).

Additionally, the tracking device or indicia may be in the form of a radio frequency identification (RFID) device built into the lobes 40, 50 of tag 20, or built into any other portion of tag 20. Such an RFID device can transmit a radio frequency signal to an RFID transceiver that can obtain the identifying information of the medical device stored in the RFID device. Additionally, the indicia can include, for example, human readable information and/or data that may include visual alphanumeric characters and tactile features such as different surface textures and/or raised or lowered portions. Furthermore, the tag 20 can include a sealable groove, slot, or compartment (not shown) that has a transparent cover such that any human and/or computer readable information can be placed into the sealable groove, slot, or compartment, but can still be read through upper planar surface 28. In addition, the indicia may include a printed adhesive label in either human and/or computer readable form that is resistant to degradation during sterilization procedures.

Furthermore, although the indicia are shown as two separate types of tracking devices, the identifying information contained within these indicia may contain the same amount of identifying information. However, it is also possible that one indicia may provide more identifying information than the other indicia. Additionally, there may be only one indicia on either lobe 40 or lobe 50, but not the other lobe. Furthermore, the same type of indicia may be represented on both lobes 40 and 50. Even more, lobes 40 and 50 may have only one indicia that spans consecutively across the enlarged substantially planar surface 60.

The embodiment shown in FIG. 1 is just one of many types of data carriers or tags that are capable of capturing and/or encapsulating a medical device, and more specifically a small device onto which identifying information typically is difficult to apply. For example, the tag may be similar to that shown in U.S. Patent Application Publication No. 2008/0230423 (the “'423 publication”). The tag described in the '423 publication provides for engagement with a storage device or other carrying or packing device, by use of a deformable snap fit engagement. The implant can be removed from the tag by use of a tool. Other tags encapsulating implants are disclosed in, for example, U.S. patent aplication Ser. No. 12/109,517, filed Apr. 25, 2008, U.S. patent application Ser. No. 12/109,534, filed Apr. 25, 2008, U.S. patent application Ser. No. 12/109,539, filed Apr. 25, 2008, and U.S. patent application Ser. No. 12/512,274, filed Jul. 30, 2009, and U.S. patent application Ser. No. ______, entitled: “Encapsulated Data Carrier Tag for Track and Trace Purposes,” docket number P00.41164, the disclosures of each of which are entirely incorporated herein by reference. Any of the aforementioned tags can be used in accordance with the embodiments described herein.

As stated above, tag 20 preferably includes a tamper evident locking mechanism so that tag 20 can be attached to and removed from a medical device 30 with no component of tag 20 remaining permanently attached to the medical device 30. Furthermore, it is preferred that there are no loose portions or particulates of tag 20 that separate from the data carrier upon unlocking the tamper evident locking mechanism. Therefore, the tamper evident locking mechanism allows tag 20 to be attached to a medical device 30 as a one-piece tag and be separated from the medical device as a one-piece tag.

The tags disclosed herein are only temporarily attached to the medical device and are utilized for tracking the use of and associated inventory levels of the medical devices. In that regard, prior to use of a medical device having such a tag associated therewith, the tag is inspected for detectable evidence of the tamper evident locking mechanism being broken and/or the tag being removed from the medical device. If there are no signs of the tag being removed from the implant and/or the tamper evident locking mechanism being broken, then a healthcare provider can remove the tag from the implant by breaking the tamper evident locking mechanism thereby releasing the medical device from the tag. If there are signs that the tamper evident locking mechanism has been tampered with and/or other evidence that the tag may have been removed from the medical device then the healthcare provider may elect not to use the medical device.

Prior to use of the medical device the tags are capable of having indicia readable by a computer. The computer readable indicia, such as a two dimensional barcode or RFID stored data can be scanned to create a label containing the identifying information relating to the medical device captured by the tag. In the event that a tag cannot be scanned, the human readable indicia on the tag can be utilized to create the label. Subsequently, the label may be attached to a patient's medical chart. Thereafter, in accordance with the preferred embodiments described herein, the tag is placed in a storage device to verify the position in which the medical device was implanted (e.g., which bone contains the implant, or what tissue contains the implant, where that bone or tissue is on the body, what side of the spine, what vertebral level, etc.). In this manner the tags can be utilized to track implant use and associated inventory levels.

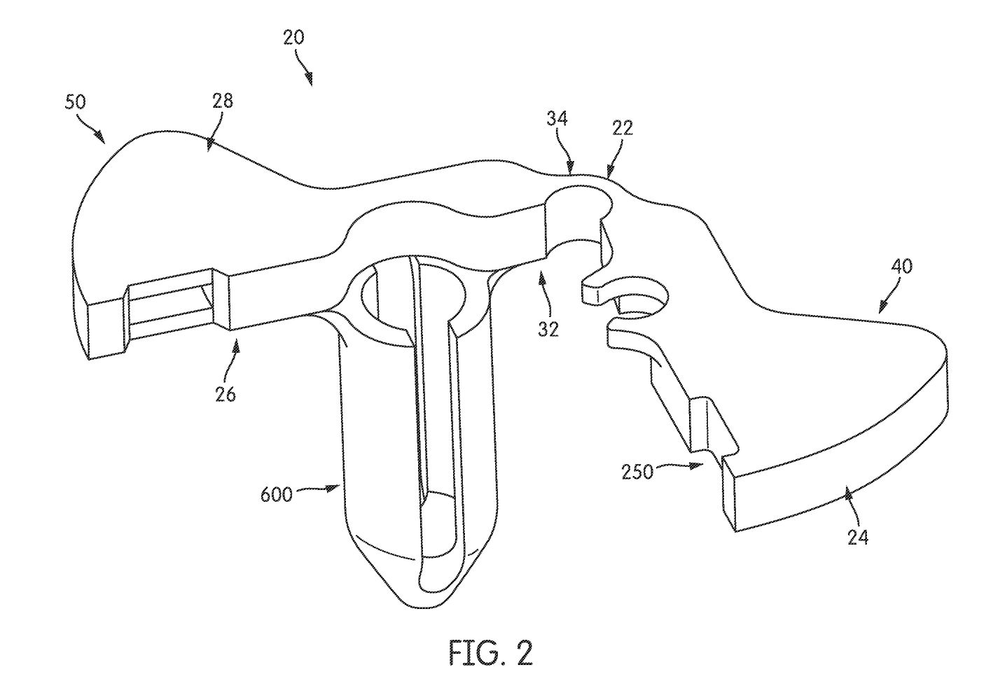

FIG. 2 illustrates another tag 20 in accordance with an additional embodiment. In this embodiment, the medical device (not shown), can be wholly or partially encapsulated via extending encapsulating portion 600. The tag 20 shown in FIG. 2 is shown in its opened position, after the medical device has been removed and the tamper evident locking mechanism has been broken at 250. Once broken and separated from the encapsulated medical device in this manner, tag 20 cannot be used again. The present invention provides a system and method for arranging the used tags 20 in a storage device so that the precise placement of each medical device can be accurately tracked, inventoried, and the location of each device can readily be scanned and stored in a data base, as welt as the patient's chart.

The system and method of a preferred embodiment encompasses removing the medical device 30 from tag 20 and implanting the medical device or otherwise making use of the medical device. If the medical device were not implanted or was found to be defective or otherwise not useable, or reusable, the device 30 and tag 20 would be discarded. On the other hand, if the medical device were implanted into a patient, the tag 20 previously associated therewith would be placed in a storage device in a position indicative of the position in which the previously associated device were implanted in the patient's body. The storage device may be shaped similarly to the area of the body where the medical device were implanted (e.g., shaped like a hip, shoulder, knee, spine, etc.), and the various bones and other tissues into which the medical device would be implanted are marked on the storage device. Hence, after use the tags 20 can be placed in the storage device, and then the entire storage device scanned after the procedure to provide an accurate record of where each medical device were implanted in the patient.

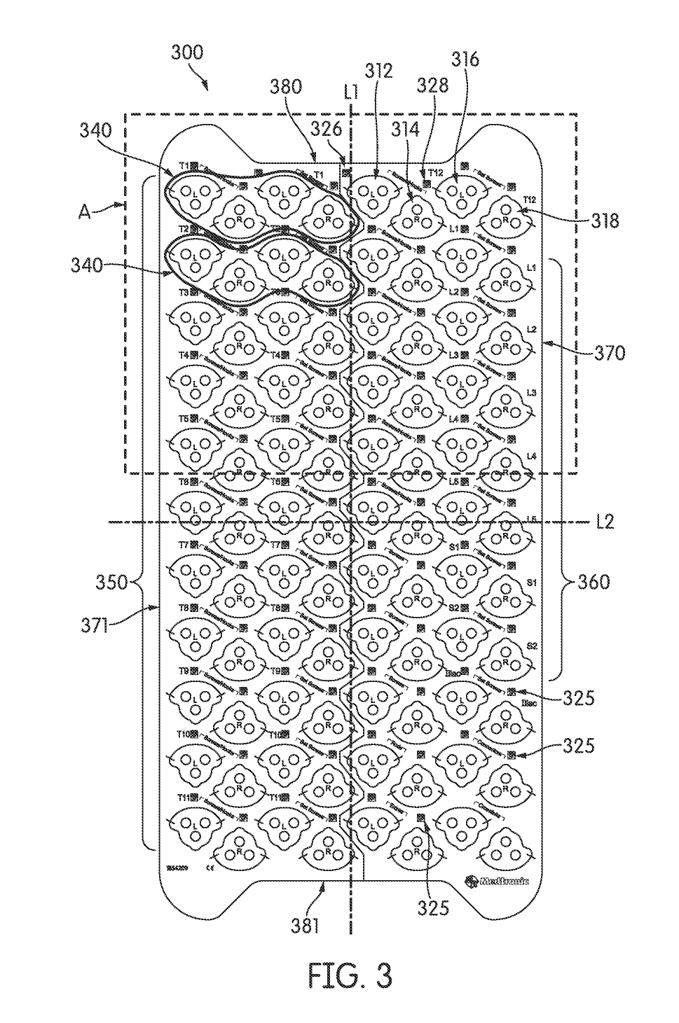

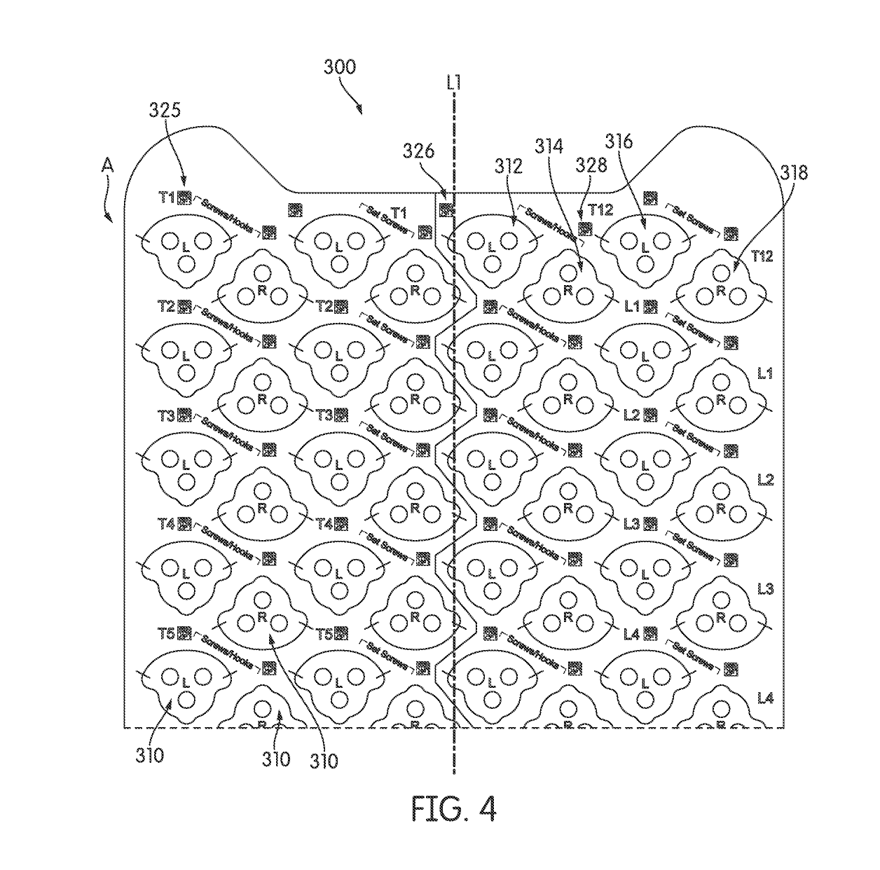

A top view of an exemplary storage device 300 is illustrated in FIG. 3. An enlarged top view of portion A of FIG. 3 is shown in FIG. 4. Like numerals represent like embodiments, and reference may be made to both FIGS. 3 and 4 for an understanding of this particularly preferred embodiment. As shown in FIG. 3, storage device 300 may be similar in many respects to a surgical tray, and it is preferred that storage device 300 be comprised of a material capable of withstanding the rigors of sterilization. Storage device 300 is shown with longitudinal axis L1 and lateral axis L2, the device 300 preferably having a pair of laterally extending front and back edges 380, 381, and a pair of longitudinally extending right and left side edges 370, 371, respectively. The storage device 300 depicted in FIG. 3 is configured for use with a spinal surgery, and more specifically, for treating a spinal deformity, which typically requires the implantation of many medical devices, and more specifically, implantation of many surgical screws and hooks, etc. The storage device 300 therefore is arranged to accept a plurality of tags 20 that can be placed in the position in which the medical device 30 previously associated with the tag 20 was implanted into the patient.

In a preferred embodiment, the storage device 300, when used to store tags 20 from a spinal implant procedure used to correct a large deformity in the thoracic and lumbar region. This type of procedure may include the implantation of long rods along the spinal column in which the rods are attached to the spinal column via multiple set screws and hooks at each level. One type of procedure may include implantation of one or more rods along multiple levels of the spine to correct a severe deformity caused by scoliosis. The storage device 300 illustrated in FIG. 3 is particularly useful for such a procedure because it enables the healthcare provider to store multiple tags 20, previously associated with pedicle screws, set screws, hooks, and other small medical devices used to connect the rod to the spinal column. The tags 20 can conveniently be stored in a plurality of pockets 310 that are of sufficient size and shape to receive the tags 20 (FIG. 3 illustrates pockets 310 of the same shape as the closed tag 20 shown in FIG. 1, after medical device 30 has been removed therefrom). While storage device 300 illustrated in FIG. 3 is a preferred device for a particular type of surgery, those skilled in the art will appreciate that any storage device 300 can be made to accommodate many different types of surgeries that involve implantation of multiple medical devices (e.g., hip, shoulder, and knee replacements, treatment of severe bone fractures in the upper and lower extremities, organ transplant surgeries, and the like)

The storage device illustrated in FIG. 3 includes a thoracic section 350 and a lumbar section 360. The storage device 300 includes a series of arranged pockets 310, each corresponding to a specific type of medical device 30 and the particular level and side of the spinal column in which the device is implant. Each level 340 of the thoracic and lumbar region includes left and right-hand side pockets 310 to accommodate tags 20 whose previously associated medical devices were implanted into the right or left hand side of the spine. For example, the first level 340 in the upper left hand corner of FIG. 3 represents the T1 level of the thoracic region of the spine. In this level 340, there are a plurality of pockets 310 marked L for left and R for right to accommodate tags 20 previously associated with screws, hooks, set screws, and the like. The plurality of pockets 310 may include pockets at each level in the spine 340, so that medical devices implanted on the left hand side of the spine on the respective vertebral bodies can be placed in their respective pockets 310. This can be seen with reference to pockets 312, 314, 316, and 318. These pockets are shown at the T12 level and represent pocket 312 to accommodate tags 20 associated with screws or hooks implanted on the left hand side of T12, and pocket 314 to accommodate tags associated screws or hooks implanted on the right hand side of T12. Similarly, pocket 316 may accommodate tags 20 associated with set screws implanted on the left hand side of T12, and pocket 318 may accommodate tags 20 associated with set screws implanted on the right hand side of T12. Similar pockets can be found along the length of the vertebral column for each vertebral level.

The storage device 300 shown in FIG. 3 may include, for example, pockets 310 that accommodate tags 20 previously associated with bone anchors. Each pocket preferably is readily identifiable by a healthcare provider, and in a most preferred embodiment, each pocket is sized and shaped to accept a used tag and has computer readable indicia 325, such as a two dimensional barcode or RFID stored data that indicates the position on the anatomy. For example, pocket 312 would have indicia 326 indicating the left hand side of the vertebral column, for a T11 vertebral body. Pocket 314 would have indicia 328 indicating the right hand side of the vertebral column for a T11 vertebral body. The indicia also may indicate the positioning on the vertebral body, such as the pedicle, spinous process, facet (superior or inferior), and the like, as well as any other identifiable information that would be desirable or helpful in determining the precise positioning of each device. After a complex surgical procedure in which multiple medical devices are implanted into a variety of positions on the spine, for example, and in which the tags 20 have been positioned in the corresponding pocket, the entire storage device 300 can be scanned and information regarding the precise location of each medical device can be stored and recorded in a patient's medical chart. For example, if the storage device 300 included two pockets 310 having positioned therein used storage tags 20, the information from the storage tags would be read and matched to the corresponding information from the indicia on the respective pocket in which it rests so that the identity of each device implanted can be determined, together with its precise location in the patient.

While not shown, the storage device 300 also may include a plurality of pockets to accommodate tags 20 associated with medical devices other than bone screws, set screws, hooks, and the like, including any other medical devices that might be used in a particular procedure. For a surgical procedure correcting a spinal deformity, for example, these pockets might accommodate tags 20 previously associated with one or more medical devices selected from the group consisting of additional bone anchors or screws that might not otherwise be anchored into a vertebral body, a spinal plate, a surgical rod, an interbody spinal device, a vertebral disc arthroplasty device, a nucleus replacement device, a corpectomy device, a vertebrectomy device, a mesh device, a facet fixation or arthroplasty device, a structural bone graft, a staple, a tether of synthetic material or wire, or other spinal fixation instrumentation. Each tag 20 previously associated with its respective medical device (e.g., plate, rod, interbody device (fusion or otherwise)), would be positioned in its respective pocket, which like pockets 310, 312, 314, 316, and 318, has computer readable indicia 325, such as a two dimensional barcode or RFID stored data that indicates the position. For example, if tag 20 were associated with an interbody device implanted between the L1 and L2 vertebral bodies, the tag 20 would be placed in a pocket corresponding to the disc space between L1 and L2. In addition, if tag 20 were associated with a rod that was implanted on the left hand side of the vertebral column along T12-L5, then tag 20 would be placed in a pocket corresponding to the left hand side of the vertebral column for T12-L5 vertebral bodies. The same procedure would apply for procedures in the thoracic section and the cervical section.

In the embodiment shown in FIG. 3, the indicia 325 may represent a two dimensional bar code and alphanumeric lettering, respectively used to identify the medical device 30 captured by tag 20 that is to be placed within the respective pocket 310. However, the indicia 325, or any tracking device herein, may be any device that is capable of retaining identifying information. For example, the indicia can be a one or two dimensional barcode capable of being scanned by an optical scanner. Such an optical scanner may include a barcode scanner made by Baracoda such as the Evolution scanner (part number: B40160202).

Additionally, the tracking device or indicia 325 may be in the form of a radio frequency identification (RPM) device built into the storage device 300. Such an RFID device can transmit a radio frequency signal to an RFID transceiver that can obtain the identifying information of the medical device stored in the RFID device. Additionally, the indicia 325 can include, for example, human readable information and/or data that may include visual alphanumeric characters and tactile features such as different surface textures and/or raised or lowered portions. Furthermore, the storage device 300 may include a sealable groove, slot, or compartment (not shown) that has a transparent cover such that any human and/or computer readable information can be placed into the sealable groove, slot, or compartment, but can still be read through the surface of the storage device 300. In addition, the indicia may include a printed adhesive label in either human and/or computer readable form that is resistant to degradation during sterilization procedures.

Those skilled in the art will appreciate that storage device 300 may have any shape or size, depending on the particular surgical procedure. For example, storage device 300 may be rectangular, oval, circular, or may have a complex shape for other surgical procedures in which multiple medical devices are implanted into a patient, including without limitation, knee, hip, and shoulder replacement surgeries, upper and lower extremity fracture fixation, reconstructive surgeries in the jaw, face, and skull, and the like. Multiple pockets 310 would be positioned on the storage device 300 to correspond to a position on the patient to make it easy for the healthcare provider to place each tag 20 previously associated with an implanted medical device in its proper position. For a hip replacement surgery, there may be a plurality of pockets to accommodate tags 20 previously associated with an acetabular cup, polyethylene liner, fixation screws, femoral stem, and the like as will be appreciated by those skilled in the art.

The tags 20 disclosed herein in whole or in part may be constructed of biocompatible materials of various types including metals or polymers. For example, the tags 20 may be made in whole or in part of a polymer known as Radel R. In such a scenario, a tag 20 is formed in whole or in part using Radel R polymer mixed with barium sulfate (BaSO₄). In this manner, the tag 20 is radiopaque such that incase of accidental implantation the tag can be located via an x-ray, for example. Further, examples of tag materials include, but are not limited to, non-cobalt-chromium alloys, titanium alloys, nickel titanium alloys, and/or stainless steel alloys, plastics and polymers including without limitation any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE.

Although the tags 20 described herein are shown associated with a bone screw 30 (FIGS. 1 and 2) this is no way implies a limitation of such medical devices the tags can be associated with and used to track. The medical device capable of being tracked by the tags described herein may be any implant or instrument used in a medical procedure. In that regard, the medical device of some embodiments may be, without limitation, a surgical screw of any variety, a spinal or other orthopedic plate, a surgical rod, an interbody spinal device, a vertebral disc arthroplasty device, a nucleus replacement device, a corpectomy device, a vertebrectomy device, a mesh device, a facet fixation or arthroplasty device, a structural bone graft, a staple, a tether of synthetic material or wire, or other spinal fixation instrumentation, an intramedullary nail, an external fixation device, a hip prosthesis or therapeutic device, a knee prosthesis or therapeutic device, or an instrument useful with any of the previously recited devices.

The tags 20 and indicia, storage devices 300, and medical devices 30 described herein are capable of undergoing one or more steam sterilization cycles, or other sterilization procedures such as radiation or gas sterilization, without degrading in a manner that would make the tag unusable and the implant unsuitable for use in a medical procedure. Therefore, the medical devices can be shipped non-sterilized from the manufacture to a healthcare provider, but the non-sterile medical device can still be tracked for inventory purposes and uses thereof through the use of the tags described herein.

The medical device 30 of this or any other embodiment of the invention may consist of materials, by way of example, and without limitation, including titanium and its alloys, ASTM material, cobalt chrome, tantalum, ceramic, poly-ether-ether-ketone (PEEK), PEAK, various plastics, plastic composites, carbon fiber composites, coral, allograft, autograft, zenograft, and can include artificial materials which are at least in part bioresorbable, or any material suitable for human implantation.

In accordance with an embodiment, the system described with reference to FIGS. 1-4 can be used to accurately track a medical device from its point of manufacture to its precise location within a patient's body. The method of tracking such a device can include, for example, prior to implantation into a patient, scanning a tag associated with a medical device and having a tamper evident locking mechanism, and removing the medical device from the tag by breaking the tamper evident locking mechanism. The method also includes providing a storage tray having a plurality of pockets to accommodate a plurality of tags previously associated with a medical device to be implanted in a patient, each pocket having computer readable indicia indicative of a position in a patient's anatomy. The method further includes placing a tag previously associated with a medical device that has been implanted in a patient in a pocket in the storage device the corresponds with the position in which the medical device was implanted. The method concludes by scanning the storage device with one or more tags positioned in its respective pocket and updating the data file to indicate where the medical device was implanted in the patient.

The storage device 300 typically is packaged together with a surgical tray or trays useful for a particular operation. By way of example only, the system and method will be described with reference to a procedure used to correct a large spinal deformity in the thoracic and lumbar region of the spine in which a rod is implanted along the T12-L5 vertebral bodies. Prior to the surgical procedure, surgical trays containing medical devices typically used in such a deformity correction procedure, and a storage device 300 capable of storing tags associated with those medical devices would be received by a healthcare provider. The trays and storage devices 300 then can be scanned and the information regarding each medical device stored into a database. Information regarding each medical device will include its prior history, such as where it was manufactured, its lot number, its part number, whether it was previously sterilized as part of a prior surgical procedure but not used, etc. The database will be updated to indicate that the surgical trays and storage devices 300 have been received by the healthcare provider and this information can be transmitted to the respective medical devices, trays, storage devices, etc.

During the surgical procedure, the healthcare provider would open the surgical tray and remove medical devices 30 from their associated tags 20 as needed. If a medical device 30 is removed from tag 20 and then not subsequently implanted, either because it was defective, or improperly sized, the device 30 and tag 20 can be placed in a position on the storage device 300 indicative of un-used but exposed medical devices. The information regarding the device can be updated in a subsequent scan. Turning now to the surgical procedure, the healthcare provider may select screws, set screws, hooks, and rods to be implanted along the T12-L5 vertebral bodies of the spine. The tags associated with each screw, hook, set screw, rod, etc., can be placed in a respective pockets 310, depending on the vertebral body (T12, L1, L2) and side of the vertebral column (left or right). The tags associated with the rod and other devices could be placed adjacent storage device 300, or placed in a pocket (not shown) in storage device 300 for such a used tag. Upon completion of the surgical procedure, the surgical trays and storage tray 300 can be scanned and reconciled to ensure that each medical device 30 previously associated with a surgical tray is either still in the tray and attached to its respective tag 20, or has been implanted and its precise location in the patient's body determined by its position in storage device 300.

To the extent a medical device 30 were removed from its associated tag 20, but not used for some reason, this information would be readily identifiable from the scan, and information regarding the un-used implant could be entered into the database (e.g., defective, or not the right size, etc.) and entered into the computer readable indicia on the respective tag. If an error is detected by virtue of a missing tag 20, for example, the patient can be scanned prior to closing to determine whether the tag was inadvertently implanted, or the area can be scanned to locate the missing tag. Data indicating the type of device, the manufacture, lot number, its position in the patient, etc. can be stored and entered into the patient's medical file, and stored in a managed database for later retrieval should a revision surgery be necessary, or for other reasons. The system and method of the preferred embodiments provide an easy and accurate mechanism to track medical devices from the point of manufacture to implantation into a patient, and can provide accurate information regarding the precise location in a patient's body where each medical device was implanted.

White the present invention has been illustrated by the above description of embodiments, and while the embodiments have been described in some detail, it is not the intention of the applicant to restrict or in any way limit the scope of the invention to such detail. Additional advantages and modifications will readily appear to those skilled in the art. Therefore, the invention in its broader aspects is not limited to the specific details, representative apparatus and methods, and illustrative examples shown and described. Accordingly, departures may be made from such details without departing from the spirit or scope of the applicant's general or inventive concept. It is understood that all spatial references, such as “longitudinal axis,” “horizontal,” “vertical,” “top,” “upper,” “lower,” “bottom,” “left,” and “right,” are for illustrative purposes only and can be varied within the scope of the disclosure.

What is claimed is:

1. A system for tracking one or more medical devices:
one or more medical device, at least one of the medical devices having associated therewith at least one tag having indicia containing information regarding the medical device to which it is associated, at least one tag comprising a tamper evident locking mechanism; and
a storage device comprising a plurality of pockets of sufficient size and shape to receive at least one tag after it has been separated from the medical device to which it was previously associated,
wherein the plurality of pockets are arranged to correspond to the position in the anatomy where the medical device is intended to be implanted, and wherein each pocket is equipped with indicia to indicate the position in the anatomy where the medical device is intended to be implanted.

2. The system of claim 1, wherein the tag comprises a material capable of undergoing sterilization without degradation such that the indicia remains readable after sterilization of the tag, the indicia includes at least one of a human readable and computer readable form.

3. The system of claim 1, wherein the indicia on the tag provides identifying information relating to at least one of the medical device, a patient receiving the medical device, a medical procedure used with the medical device, and a material and process of manufacturing associated with the medical device.

4. The system of claim 1, wherein the tamper evident locking mechanism provides an unlocked position for receiving a medical device between the first and second lobes and a locked position in which the first and second lobes are locked together preventing the medical device from being released from the tag without detectable evidence.

6. The system of claim 1, wherein the storage device further comprises laterally extending front and back edges, longitudinally extending right and left sides, a plurality of pockets arranged along a left side of the storage device, a plurality of pockets arranged along the right side of the storage device, and at least one pocket positioned centrally adjacent the front edge, at least one pocket positioned centrally adjacent at least the right and left side edge, and at least one pocket positioned centrally adjacent the back edge.

7. The system of claim 6, wherein the plurality of pockets arranged along the left side of the storage device receive tags previously associated with medical devices implanted on the left side of a vertebral column of a patient, and the plurality of pockets arranged along the right side of the storage device receive tags previously associated with medical devices implanted on the right side of a vertebral column of a patient.

8. The system of claim 7, wherein the plurality of pockets arranged along the left side and the right side of the storage device are positioned to correspond to each vertebral level in the spinal column.

9. The system of claim 1, wherein the medical device is selected from the group consisting of a surgical screw, a spinal plate, an orthopedic plate, a surgical rod, an interbody spinal device, a vertebral disc arthroplasty device, a nucleus replacement device, a corpectomy device, a vertebrectomy device, a mesh device, a facet fixation or arthroplasty device, a structural bone graft, a staple, a tether of synthetic material or wire, an intramedullary nail, an external fixation device, a hip prosthesis, a knee prosthesis, and combinations thereof.

10. The system of claim 1, wherein the one or more medical device and tag associated therewith, and the storage device are comprised of a material capable of undergoing one or more sterilization procedures without degrading in a manner that would make the medical device, tag, or storage device unusable or unsuitable for use in a medical procedure.

11. A method for tracking one or more medical devices comprising:
providing one or more medical devices, at least one of the medical devices having associated therewith at least one tag having indicia containing information regarding the medical device to which it is associated, at least one tag comprising a tamper evident locking mechanism;
providing a storage device for storing a plurality of tags, the storage device comprising a plurality of pockets of sufficient size and shape to receive at least one tag after it has been separated from the medical device to which it was previously associated, wherein the plurality of pockets are arranged to correspond to the position in the anatomy where the medical device is intended to be implanted, and wherein each pocket is equipped with indicia to indicate the position in the anatomy where the medical device is intended to be implanted;
reading the indicia on the at least one tag prior to a surgical procedure and entering the data read from the at least one tag into a database;
separating at least one medical device from its associated tag;
placing the tag previously associated with the at least one medical device in the storage device in one of the plurality of pockets in the storage device corresponding to the position in the anatomy where the at least one medical device was implanted; and
reading the indicia on the plurality of pockets and the indicia on the at least one tag placed in the pocket and entering the data read into a database to correlate the information concerning each medical device with its position in the anatomy.

13. The method of claim 12, further comprising updating a patient record to indicate where each medical device was implanted in the patient.

14. The method of claim 12, wherein the indicia on the tag provides identifying information relating to at least one of the medical device, a patient receiving the medical device, a medical procedure used with the medical device, and a material and process of manufacturing associated with the medical device.

15. The method of claim 12, wherein the tamper evident locking mechanism provides an unlocked position for receiving a medical device between the first and second lobes and a locked position in which the first and second lobes are locked together preventing the medical device from being released from the tag without detectable evidence.

16. The method of claim 12, wherein the storage device further comprises laterally extending front and back edges, longitudinally extending right and left sides, a plurality of pockets arranged along a left side of the storage device, a plurality of pockets arranged along the right side of the storage device, and at least one pocket positioned centrally adjacent the front edge, at least one pocket positioned centrally adjacent at least the right and left side edge, and at least one pocket positioned centrally adjacent the back edge.

17. The method of claim 16, wherein the plurality of pockets arranged along the left side of the storage device receive tags previously associated with medical devices implanted on the left side of a vertebral column of a patient, and the plurality of pockets arranged along the right side of the storage device receive tags previously associated with medical devices implanted on the right side of a vertebral column of a patient.

18. The method of claim 17, wherein the plurality of pockets arranged along the left side and the right side of the storage device are positioned to correspond to each vertebral level in the spinal column.

19. The method of claim 12, wherein the medical device is selected from the group consisting of a surgical screw, a spinal plate, an orthopedic plate, a surgical rod, an interbody spinal device, a vertebral disc arthroplasty device, a nucleus replacement device, a corpectomy device, a vertebrectomy device, a mesh device, a facet fixation or arthroplasty device, a structural bone graft, a staple, a tether of synthetic material or wire, an intramedullary nail, an external fixation device, a hip prosthesis, a knee prosthesis, and combinations thereof.

20. The method of claim 12, wherein the one or more medical device and tag associated therewith, and the storage device are comprised of a material capable of undergoing one or more sterilization procedures without degrading in a manner that would make the medical device, tag, or storage device unusable or unsuitable for use in a medical procedure.
